SEQUENCE LISTING (2 sequences)

SEQ ID NO: 1 - protein, 10 aa, Human
GGRRNNYYCC

SEQ ID NO: 2 - DNA, 43 bp, Artificial
  Oligonucleotide
attattattattcaaatggatttatttatttatttatttattt

(19) United States
(12) Patent Application Publication
(10) Pub. No.: US 2020/0046671 A1
(43) Pub. Date: Feb. 13, 2020

(54) APOBEC3B MUTAGENESIS AND IMMUNOTHERAPY

(71) Applicant: Regents of the University of Minnesota, Minneapolis, MN (US)

(72) Inventor: Reuben S. Harris, St. Paul, MN (US)

(21) Appl. No.: 15/738,658

(22) PCT Filed: Jun. 29, 2016
(86) PCT No.: PCT/US16/40032

§ 371 Date: Dec. 21, 2017

Related U.S. Application Data
(60) Provisional application No. 62/186,228, filed on Jun. 29, 2015.
(60) Provisional application No. 62/187,623, filed on Jul. 1, 2015.

(51) Int. Cl. A61K 31/23; A61P 35/00; A61K 39/395; G01N 33/574; A61K 9/00

(52) U.S. Cl. CPC A61K 31/23; A61P 35/00; A61K 9/0019; G01N 33/57484; A61K 39/39558

(57) ABSTRACT

A method of treating a subject having a tumor generally includes administering to the subject an amount of an APOBEC3 upregulator effective to increase mutagenesis in cells of the tumor.

### CROSS-REFERENCE TO RELATED APPLICATION

This application claims priority to U.S. Provisional Patent Application No. 62/186,228, filed Jun. 29, 2015, and U.S. Provisional Patent Application No. 62/187,623, filed Jul. 1, 2015, each of which is incorporated herein by reference.

### SEQUENCE LISTING

This application contains a Sequence Listing electronically submitted via EFS-Web to the United States Patent and Trademark Office as an ASCII text file entitled “2016-06-27-SeqListing_ST25.txt” having a size of 1 kilobytes and created on Jun. 27, 2016. The information contained in the Sequence Listing is incorporated by reference herein.

### SUMMARY

This disclosure describes, in one aspect, a method of treating a subject having a tumor.

Generally, the method includes administering to the subject an amount of an APOBEC3 upregulator effective to increase mutagenesis in cells of the tumor.

In some embodiments, the APOBEC3 upregulator is co-administered with an immunotherapy. In some of these embodiments, the APOBEC3 upregulator is administered prior to the immunotherapy. In other of these embodiments, the immunotherapy can include administering to the subject a therapeutic molecule that blocks an immune checkpoint.

In some embodiments, the method can further include obtaining a sample from the tumor and determining whether tumor cells express an APOBEC3 mRNA, an APOBEC3 polypeptide, or an APOBEC3 mutation signature.

In some embodiments, the APOBEC3 upregulator is administered intratumorally. In other embodiments, the APOBEC3 upregulator is administered systemically.

In some embodiments, the APOBEC3 upregulator is administered via functionalized liposomes. In other embodiments, the APOBEC3 upregulator is administered via a virus-based vector.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. APOBEC3B upregulation by PMA. (A) A histogram showing the specific upregulation of APOBEC3B mRNA by PMA. MCF10A cells were treated with PMA (25 ng/ml) or vehicle control for six hours, and mRNA levels were measured by RT-qPCR (mean and SD are shown for triplicate RT-qPCR reactions normalized to TBP). The same data points are shown in the context of a larger PMA dose response experiment in FIG. 6. (B) A histogram demonstrating the dose responsiveness of APOBEC3B upregulation by PMA. Normalization and quantification were calculated as in FIG. 1A. The middle images show immunoblots for corresponding APOBEC3B and tubulin proteins levels, and the lower image shows DNA cytosine deaminase activity for the corresponding whole cell extracts (S, substrate; P, product; percent deamination quantified below each lane). (C) A histogram depicting the rapid kinetics of APOBEC3B upregulation following PMA treatment. MCF10A cells were treated with a single concentration of PMA (25 ng/ml), and mRNA, protein, and activity levels are reported as in FIG. 1B. (D) New protein synthesis is dispensable for APOBEC3B mRNA upregulation by PMA. Representative dose response experiment for MCF10A cells treated with the indicated concentrations of PMA following a 30-minute pretreatment with 10 μg/mL cyclohexamide. mRNA, protein, and activity levels are reported as in FIG. 1B.

FIG. 2. APOBEC3B upregulation by PMA is dependent on PKC. (A)-(F) Histograms reporting the impact of the indicated small molecules on PMA-induced APOBEC3B upregulation. APOBEC3B induction was inhibited by Go6983 (pan-PKC inhibitor), BIM-1 (classical and novel PKC inhibitor), Go6976 (classical PKC selective inhibitor), and AEB071 (preclinical PKC inhibitor), but not by LY294002 (PI3K inhibitor) or UO126 (MEK inhibitor). MCF10A cells were treated with PMA following a 30-minute pretreatment with the indicated concentrations of each inhibitor. mRNA expression is reported as the mean of three independent RT-qPCR reactions normalized to TBP (error bars report SD from triplicate assays). (G) Histogram depicting PKC isoforms expressed in MCF10A cells treated with PMA or vehicle control. mRNA expression was determined by RNA-seq and is reported as fragments per kilobase of exon per million fragments mapped (FKPM) and normalized to TBP. (H) Histogram showing that PKCa knockdown inhibits APOBEC3B induction by PMA. MCF10A cells were treated with PMA following PKCa knockdown using three independent PKCa specific shRNA encoding lentiviruses and a control. mRNA levels for both PKCa (blue) and APOBEC3B (red) are reported. (I) Immunoblots confirming PKCa knockdowns and proportional reductions in APOBEC3B protein levels.

FIG. 3. Non-canonical NFκB signaling is responsible for APOBEC3B upregulation by PMA. (A-B) Histograms depicting the dose responsive inhibition of PMA-induced APOBEC3B upregulation by BAY 11-7082 (ubiquitination inhibitor) and MG132 (proteasome inhibitor). MCF10A cells were treated with PMA following a 30-minute pretreatment with the indicated concentrations of each inhibitor. APOBEC3B mRNA expression is reported as the mean of three independent RT-qPCR reactions normalized to TBP (error bars report SD from triplicate assays). (C) Histogram depicting NFKcB subunit mRNA levels in MCF10A cells treated with PMA or vehicle control. Expression was determined by RNA-seq and is reported as FKPM and normalized to TBP. (D) Plot depicting inhibition of PMA-induced APOBEC3B expression by the IκB kinase (IKK) inhibitor, TPCA-1, near the IC50 for IKKα, not IKKβ. MCF10A cells were treated with PMA following treatment with varying concentrations of TPCA-1. TNFα (light) and APOBEC3B (dark) mRNA levels are reported as the mean of three independent RT-qPCR reactions normalized to TBP (error bars report SD from triplicate assays). The dotted lines denote previously reported in vitro IC50 values for IKKα and IKKβ inhibition by TPCA-1 (Podolin et al., 2005, J Pharmacol Exp Ther. 312:373-381). (E) Histogram showing the kinetics of NFKBIA upregulation PMA. MCF10A cells were treated with PMA for the indicated times and mRNA values were quantified as in FIG. 3A. (F) The APOBEC3B and NFKBIA promoter regions contain several putative NFκB binding sites (TSS, transcriptional start site). (G) RELB and p105/p52 are specifically and robustly recruited to the APOBEC3B promoter region by PMA. ChIP was performed after a treatment with PMA or vehicle control for two hours. qPCR results are reported as percent of the total chromatin input.

FIG. 4. The PKC-NFκB pathway drives endogenous APOBEC3B expression in cancer cells. (A) APOBEC3B mRNA levels in representative breast, ovarian, and head/neck cancer cell lines. mRNA expression is reported as the mean of three independent RT-qPCR reactions normalized to TBP (error bars report SD from triplicate assays). (B) Representative PKC inhibitor treated cancer cell line experiment. Each line was treated with AEB071 (10 μM) or vehicle control for 48 hours prior to analysis. The histogram reports APOBEC3B mRNA levels normalized to the vehicle treated control for each line. The middle images show immunoblots for corresponding APOBEC3B and tubulin protein levels, and the lower image shows DNA cytosine deaminase activity for the corresponding whole cell extracts (S, substrate; P, product; percent deamination quantified below each lane).

FIG. 5. Model for APOBEC3B upregulation by the PKC-NFκB pathway. PKCa activation by DAG or PMA leads to IKKα phosphorylation and proteasome-dependent cleavage of NFκB subunit p100 into the transcriptionally active p52 form. The non-canonical NFκB heterodimer containing p52 and RELB is then recruited to the APOBEC3B promoter to drive transcription. Red labels represent the small molecules and approaches used to interrogate this signal transduction pathway.

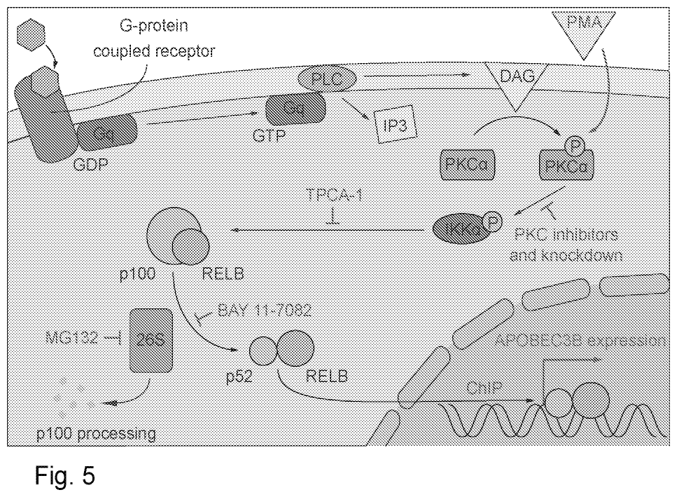

FIG. 6. APOBEC family member mRNA levels in MCF10A cells treated with the indicated PMA concentrations or DMSO as vehicle control for six hours. mRNA expression is reported as the mean of three independent RT-qPCR reactions normalized to TBP (error bars report SD from triplicate assays). The 25 ng/ml data are shown in FIG. 1A.

FIG. 7. APOBEC3B upregulation by PMA is dependent on NFκB-inducing kinase (NIK). (A) Representative dose response experiment for MCF10A cells treated with PMA following a 30-minute pretreatment with the indicated concentrations of NIK inhibitor (shown in (C); Compound 31 from Li et al., 2013, Bioorganic & Medicinal Chemistry Letters 23:1238-1244). mRNA expression is reported as the mean of three independent RT-qPCR reactions normalized to TBP (error bars report SD from triplicate assays). (B) Representative dose response experiment in which A549 cells with a canonical NFκB luciferase reporter are treated with TNF-α (canonical pathway inducer) or TNF-α with the indicated concentrations of NIK inhibitor (non-inhibitory in this assay) or parthenolide (Parth), which is a known canonical pathway inhibitor (Kwok et al., 2001, Chemistry & Biology 8, 759-766). (C) Chemical structure of NIKi (Compound 31 from Li et al., 2013, Bioorganic & Medicinal Chemistry Letters 23:1238-1244).

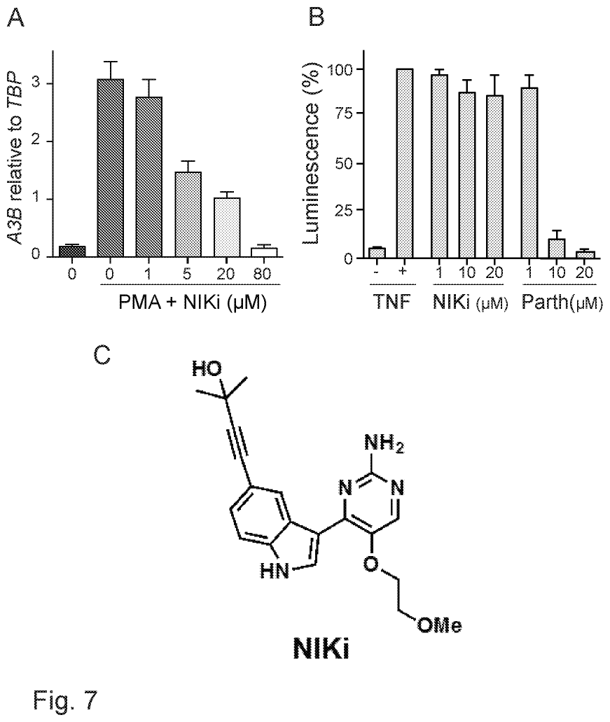

### DETAILED DESCRIPTION OF ILLUSTRATIVE EMBODIMENTS

A novel approach to increasing the durable response rate to cancer immunotherapies is to intentionally increase the mutational load in a tumor, thereby creating neoantigens and/or neoepitopes that stimulate a T cell response to the tumor. This approach involves increasing the mutation selectivity for tumor tissue and to follow the increase in tumor mutational load with at least one course of immunotherapy, which may include inhibiting an immune checkpoint with a monoclonal antibody against CTLA-4, PD-1, and/or PD-L1. Conventional methods for increasing the mutational load in a subject's tumors—e.g., smoking or increased ultraviolet (UV) light exposure—are inadvisable for therapeutic purposes. One feature of the methods described herein is inducing an increase in the mutational load in a tumor. Increasing the mutational load in cancer can make the tumor more recognizable to the subject's own immune system (e.g., cytotoxic T cells). When combined with therapies that either prime the immune system or decrease repression of the immune system, the effect of increasing mutation in tumor tissue is a more complete identification and eradication of the tumor and, thus, a more durable response to cancer immunotherapies.

APOBEC3B upregulation correlates with higher somatic mutation loads and its intrinsic DNA deamination preference matches the cytosine mutation signatures observed in many different cancers. This disclosure describes methods for APOBEC3B upregulation. Increased APOBEC3B mRNA and protein levels lead to increased DNA deamination activity, higher levels of somatic mutation, and corresponding increases in neoantigens. As used herein the term “neoantigen” refers to a peptide antigen that is expressed by, and specific to, a mutating tumor cell and can be recognized as foreign by a subject's immune system. As used herein, the term “neoepitope” refers to a region of a neoantigen that is the target of an immune response against the neoantigen. Increasing somatic mutagenesis can be used to prime existing immunotherapies by creating a larger number of tumor-specific antigens (e.g., neoantigens) for immune cells to recognize.

Thus, increasing the mutational burden of a tumor can increase the likelihood of the development of specific neoepitopes that can confer clinical benefit. In the context of immunotherapy, genetic diversity of cancer may provide a resource for tumors to develop a greater spectrum of neoepitopes and, therefore, neoantigens that can serve as immunological targets for immunotherapies.

Somatic mutations are present in many forms of cancer. Mutations happen when DNA damage escapes repair. Established sources of mutation include, for example, ultraviolet light in skin cancer, tobacco carcinogens in lung cancer, and water-mediated deamination of methyl-cytosine as a function of age in many other cancers. A more recently discovered source of mutation is the plant-derived dietary supplement aristolochic acid, which causes A-to-T transversion mutations in liver and bladder cancers. Another source of mutation is the APOBEC family of DNA cytosine deaminases, which cause signature C-to-T transition and C-to-G transversion mutations in, for example, breast, head/neck, bladder, cervical, lung, ovarian, and other cancers. Many APOBEC mutational events are dispersed throughout the genome, but a minority of APOBEC mutational events can be found in dense strand-coordinated clusters termed kataegis.

Expression profiling and functional studies independently identified APOBEC as a major source of mutation in cancer. In particular, APOBEC3B is upregulated in breast and ovarian cancer cell lines and primary tumors. APOBEC3B is predominantly nuclear, and knockdown experiments demonstrated APOBEC3B-mediated DNA cytosine deaminase activity in cancer cell line extracts. Moreover, APOBEC3B mediates elevated levels of genomic uracil and increased mutation rates. APOBEC3B levels correlate with higher C-to-T and overall base substitution mutation loads. The biochemical preference of recombinant APOBEC3B deduced in vitro closely resembled the actual cytosine mutation bias in breast cancer as well as in several of the other tumor types listed above (i.e., strong bias toward 5′-TC dinucleotides).

Human cells have the potential to express up to seven distinct antiviral APOBEC3 enzymes. Each enzyme has a biochemical preference for deaminating cytosines in single-stranded DNA, but activity is strongly influenced by flanking bases at the −2, −1, and +1 positions relative to the target cytosine.

This disclosure describes APOBEC3 upregulation by administering to a subject an effective amount of an APOBEC3 upregulator. As one example, the diacylglycerol mimic phorbol 12-myristate 13-acetate (PMA, a phorbol ester) was used as a model APOBEC3B upregulator. PKC activation through the PKC-noncanonical NFκB pathway causes specific and dose-responsive increases in APOBEC3B mRNA, protein, and activity levels, which are strongly suppressed by PKC and noncanonical NFκB inhibition. Induction correlates with RELB (but not RELA) recruitment to endogenous APOBEC3B, implicating noncanonical NFiB signaling. Relevance to tumors is supported by PKC inhibitor-mediated APOBEC3B downregulation in multiple cancer cell lines. These data establish the first mechanistic link between APOBEC3B and a common signal transduction pathway. Thus, activation of this pathway may be exploited to increase APOBEC3 expression, elevate somatic mutation rates, and provide opportunities for immunotherapies and/or synthetic lethal approaches to treat tumors.

As used herein, an APOBEC3 upregulator refers any substance that can increase APOBEC3B expression and, because this enzyme is only known to be a DNA cytosine deaminase, therefore, it will also increase the mutational load of a cell. An APOBEC3B upregulator may be, for example, a compound or other substance that increases APOBEC3B expression through molecules or substances that upregulate APOBEC3B through activation of the PKC-non-canonical NFκB pathway, an allosteric or direct interaction with the enzyme, or a compound or other substance that upregulates APOBEC3B through some other mechanism. Exemplary APOBEC3B upregulators that activate the PKC-non-canonical NFκB pathway include, for example, PMA (or other phorbol ester), agonists of the lymphotoxin-(3 receptor (LTβR), and ingenol mebutate. Exemplary LTβR agonists include, for example, the antibodies BS1 and CBE11 (Lucifora et al., 2014, Science 343:1221-1227; Lukashev et al., 2006, Cancer Res 66(19):6917-6924).

Several other methods for upregulation of APOBEC3B through the non-canonical NFκB pathway include, the use of Smac mimetics, cleaving NIK, activating IKKα, and overexpressing the P52 subunit.

Smac mimetics such as LCL-161, GDC-0152 (also known as CUCD-427), TL32711 (also known as birinapant), AT-406 (also known as Debiol 143, Debiopharm Group S.A., Lausanne, Switzerland), and HGS 1029 that result in depletion of cellular inhibitor of apoptosis (cIAP) proteins. Lowering cellular levels of cIAP proteins with Smac mimetics thereby results in less cIAP-mediated ubiquitination and proteosomal degradation of NIK, which in turns results in elevated noncanonical NFκB signaling and expression of target genes, such as APOBEC3B.

Cleaving NIK by, for example, the MALT 1 paracaspase creates a stable form of NIK that constitutively activates the noncanonical NFκB pathway.

Constitutive IKKα activation by the Tax oncoprotein of human T-cell leukemia virus type 1 (HTLV1) activates NIK, which phosphorylates Ser-176 in the activation loop of IKKα, resulting in kinase activity. Stably expressing a constitutively active (dominant) variant of IKKα (Ser-176-Asp or Glu) selectively activates the noncanonical NFκB pathway.

Overexpressing the P52 subunit, which binds RELB in the non-canonical NFκB transcription complex, also activates expression of genes such as APOBEC3B that are driven by the non-canonical NFκB transcription pathway.

Exemplary additional substances that can upregulate APOBEC3 include, for example, a genetically-modified version of a virus (e.g., HPV or Polyoma virus) that is known to upregulate APOBEC3B or a fragment thereof effective to upregulate APOBEC3B. Additionally, a CRISPR-activation approach involving the delivery of a Cas9-TA APOBEC3B promoter-specific guide RNA complex to tumor cells such that the transcriptional activator (TA) tethered to Cas9 activates APOBEC3B gene expression and mutagenesis.

### Specific Upregulation of APOBEC3B by PMA

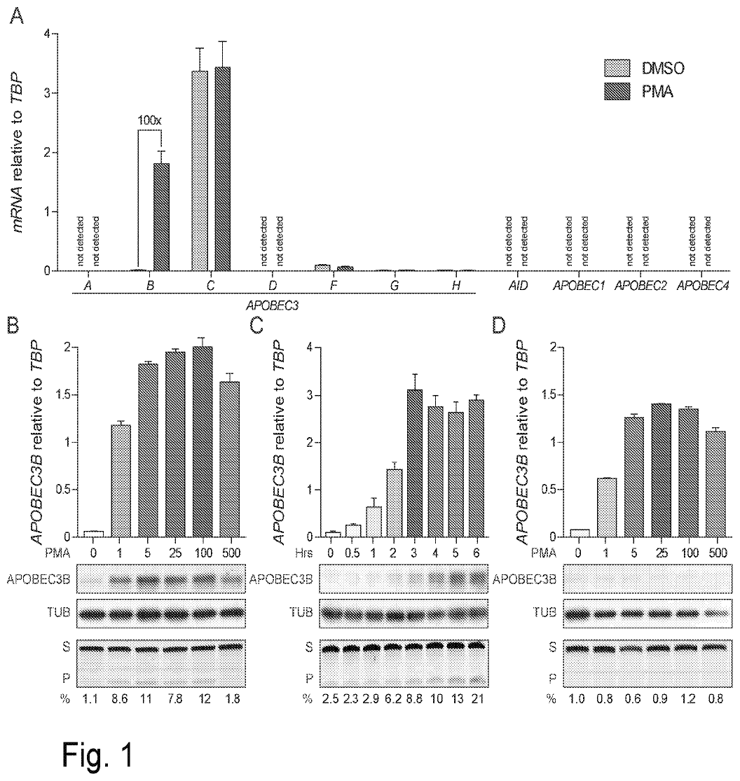

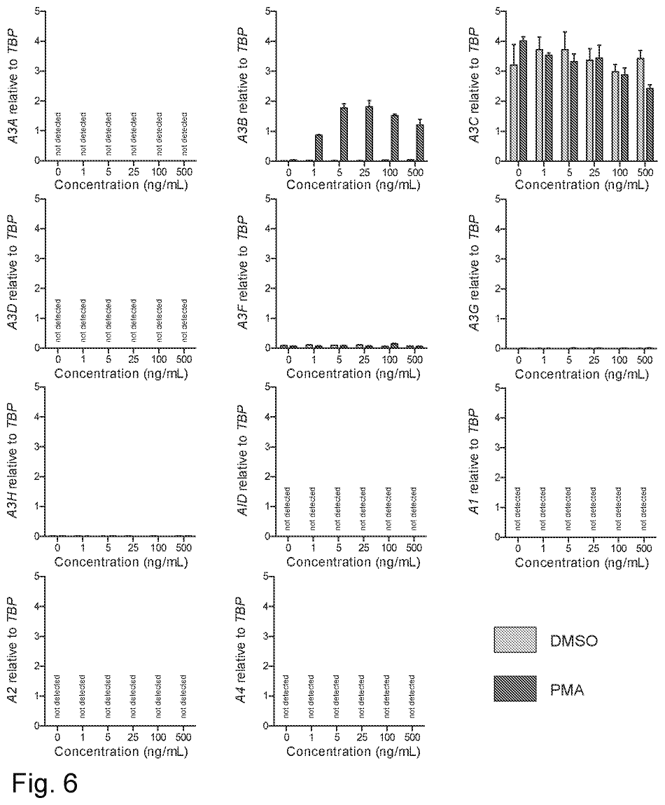

A panel of immortalized normal human epithelial cells lines and breast cancer cell lines was treated with the model PKC-NFκB pathway activator PMA (or equal amounts of DMSO as a negative control) and previously validated reverse transcription quantitative PCR (RT-qPCR) assays were used to measure mRNA levels of all eleven human APOBEC family members. APOBEC3B mRNA was induced specifically by PMA treatment of several lines including the immortalized normal breast epithelial cell line MCF10A (FIG. 6). Under standard cell culture conditions, MCF10A expresses low levels of APOBEC3B and APOBEC3F, even lower levels of APOBEC3G and APOBEC3H, high levels of APOBEC3C, and undetectable levels of all other APOBEC family members. PMA treatment caused a specific 100-fold upregulation of APOBEC3B mRNA, with no detectable changes in the expression levels of any other APOBEC family members (FIG. 1A and FIG. 6).

APOBEC3B was induced with as little as 1 ng/mL PMA, and its induction was dose responsive and near maximal at 25 ng/mL PMA (FIG. 1B, histogram). APOBEC3B mRNA levels correlated with a rise in steady-state protein levels as measured by immunoblotting with a rabbit anti-APOBEC3B monoclonal antibody (described in U.S. Provisional Patent Application No. 62/186,109, filed Jun. 29, 2015) and with enzymatic activity as measured by a gel-based single-stranded DNA cytosine deamination assay (FIG. 1B). Moreover, significant APOBEC3B mRNA induction was detected 30 minutes after PMA treatment and maximal levels were observed by three hours post-treatment (FIG. 1C, histogram). APOBEC3B protein and activity levels lagged shortly behind mRNA levels and persisted through the duration of the time course (FIG. 1C, immunoblot and polyacrylamide gel). APOBEC3B upregulation may be a direct result of signal transduction as the kinetics of upregulation were not affected by simultaneously treating cells with the protein translation inhibitor cyclohexamide (FIG. 1D). Altogether, these data demonstrate that APOBEC3B is strongly and specifically upregulated by a PMA-induced signal transduction mechanism in the immortalized normal breast epithelial cell line MCF10A. Notably, upregulation can be as high as 100-fold and this maximal level of APOBEC3B mRNA is consistent with that observed in many different tumor types including, for example, a large fraction of breast and ovarian cancers—e.g., mRNA levels 2-fold to 5-fold higher than those of the constitutively expressed housekeeping gene TBP.

### PKC is Involved in APOBEC3B Induction by PMA

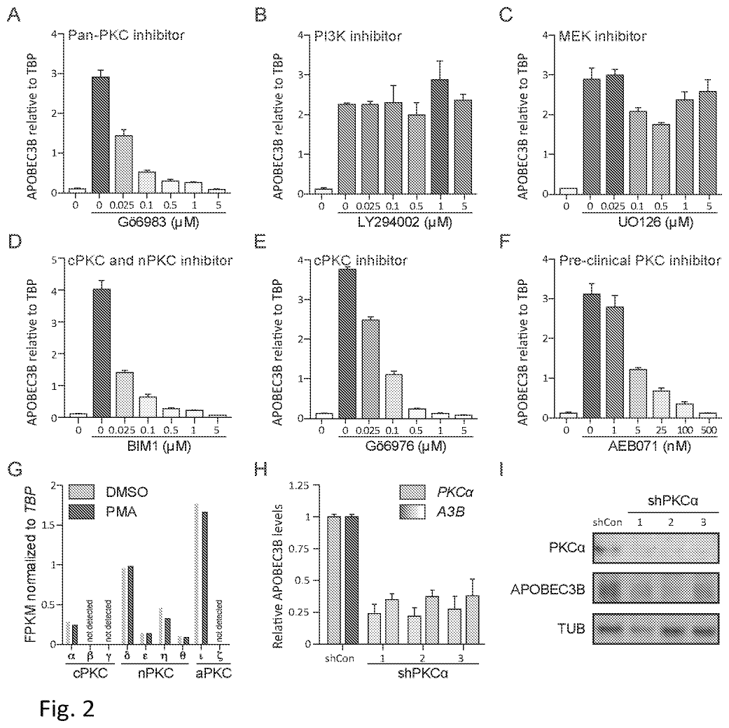

PMA is a well-known agonist of PKC, but it also affects other cellular processes. To determine whether APOBEC3B induction by PMA occurs through PKC signal transduction or an alternative mechanism, a panel of existing PKC inhibitors that vary with respect to class selectivity were analyzed. MCF10A cells were pre-treated for 30 minutes with varying concentrations of the pan-PKC inhibitor G66983 (Gschwendt et al., FEBS Lett. 392(2):77-80, 1996) and then treated for six hours with PMA (25 ng/mL). In comparison to strong APOBEC3B upregulation observed with PMA treatment alone, pretreatment with G66983 caused a dose responsive suppression of APOBEC3B induction (FIG. 2A). APOBEC3B was suppressed to background levels with 5 μM G66983, as well as higher concentrations (FIG. 2A). No morphological defects or viability issues were observed at these low concentrations of G66983 (data not shown). As additional controls, MCF10A cells were pretreated in parallel with the phosphoinositol 3 kinase (PI3K) inhibitor, LY294002, and the mitogen-activated protein kinase kinase (MEK) inhibitor, U0126, prior to PMA induction (FIGS. 2B and 2C). In both instances, no suppression of APOBEC3B upregulation was observed. Taken together, these data indicated that the PKC pathway regulates endogenous APOBEC3B expression in the MCF10A breast epithelial cell line, and the PI3K and MEK pathways are unlikely to be involved.

Human cells can express up to nine different PKC genes. The nine distinct PKC proteins (conventionally called isoforms) can be divided into three distinct classes based on activation mechanisms: classical PKC (cPKC) isoforms require both DAG and increased levels of intracellular calcium, novel PKC (nPKC) isoforms require only DAG, and atypical PKC (aPKC) isoforms are activated by other signals. Because DAG mimics do not generally activate aPKCs, the aPKC isoforms are unlikely to be involved in PMA-induced APOBEC3B upregulation. To test this, MCF10A cells were pre-treated with AEB071, which is a well characterized inhibitor of the cPKC and nPKC isoforms (but not the aPKC isoforms), and then induced with PMA and quantified APOBEC3B expression levels. Again, a clear dose dependent suppression of APOBEC3B induction was observed (FIG. 2D). Moreover, AEB071 caused complete suppression at 500 nM, which is approximately 10-fold more potent than G66983, consistent with IC50 values reported previously for this molecule (Gschwendt et al., FEBS Lett. 392:77-80, 1996; Evenou et al., J Pharmacol Exp Ther. 330:792-801, 2009; Wagner et al., J Med Chem. 54:6028-6039, 2011).

The responsible PKC isoforms were further narrowed down by pretreating MCF10A with Go6976, which is an inhibitor of the cPKC class of proteins. The dose responsiveness of APOBEC3B repression was similar to G66983 (FIG. 2E), consistent with previously reported IC50 values (Gschwendt et al., FEBS Lett. 392:77-80, 1996; Martiny-Baron et al., J Biol Chem. 268:9194-9197, 1993). Taken together, the chemical inhibition data indicate that a cPKC isoform mediates APOBEC3B induction by PMA. RNA sequencing (RNAseq) revealed that PKCα (PRKCA) is the only cPKC isoform expressed in MCF10A cells (FIG. 2F). PKCα mRNA levels were unchanged by PMA treatment, in comparison to DMSO as a negative control, further confirming that PMA binding signals directly through PKCα to ultimately stimulate APOBEC3B transcription (FIG. 2F).

Non-Canonical NFKcB Signaling is Involved with APOBEC3B Induction by PMA

Downstream transcription factors responsible for driving APOBEC3B upregulation in response to PMA were identified. PKC signals through several different transcription factors, including ERK, JNK, NFκB, and others. First, APOBEC3B promoter region were analyzed for binding sites of known PKC-regulated transcription factors. These in silico analyses revealed several NFκB binding sites within 2.5 kb of the APOBEC3B transcriptional start site (5′-GGRRNNYYCC-3′; SEQ ID NO:1).

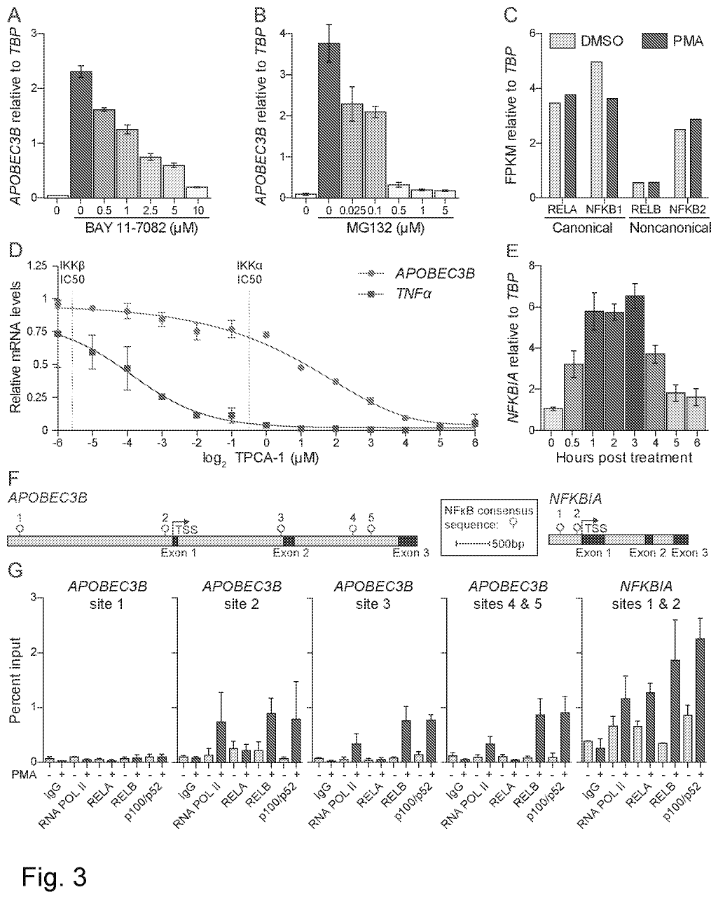

To test for a mechanistic link between NFκB and APOBEC3B transcription, MCF10A cells were pretreated with varying amounts of BAY 11-7082, which is an NFκB inhibitor that acts by inhibiting upstream IκB kinases (IKKs), and then added PMA at concentrations effective for APOBEC3B induction. BAY 11-7082 caused strong dose-responsive drops in APOBEC3B induction by PMA treatment (FIG. 3A).

The canonical and noncanonical NFκB signaling pathways involve proteasome-mediated degradation of IκB and p100, respectively, for efficient signal transduction. Therefore, degradation of these proteins was blocked by pretreating MCF10A cells with a titration of the proteasome inhibitor, MG132, prior to PMA stimulation. Under these conditions, APOBEC3B expression decreased in a dose dependent manner in response to MG132 treatment (FIG. 3B), indicating that the pathway of interest requires protein degradation by the proteasome for productive signal transduction.

RNAseq data sets revealed that MCF10A cells express both the canonical NFκB components, RELA and NFKB1, and the noncanonical NFκB components, RELB and NFKB2, and that expression levels are unaffected by PMA treatment (FIG. 3C). These data also revealed that several known NFκB regulated genes are upregulated in MCF10A cells in response to PMA. A subset of these RNAseq results was validated by RT-qPCR of PMA-upregulated NFκB target genes, including NFKBIA, which encodes IκB (FIG. 3D).

### RELB is Recruited to the APOBEC3B Promoter Region in Response to PMA

Next, a series of chromatin immunoprecipitation (ChIP) experiments were performed to determine whether the canonical or noncanonical NFκB pathway is responsible for upregulating APOBEC3B. Primer sets were designed for each of the predicted NFκB binding sites near the APOBEC3B transcriptional start site, as well as a control set in the promoter region of NFKBIA (FIG. 3E). ChIP was performed for RELA, RELB, RNA POL II (positive control), and isotype matched IgG (negative control). RELA, RELB, and RNA POL II were all bound to the NFKBIA promoter following PMA treatment (FIG. 3F). In addition, RNA POL II strongly bound to the APOBEC3B gene near the transcriptional start site (FIG. 3F). Also, RELB bound both near the transcriptional start site and at sites 4 and 5, which are located in intron 2 and too close together to be distinguished by this procedure. Binding also may occur at lower levels at site 3 in intron 1, but the IgG signal was too high to distinguish background from actual binding. These ChIP data strongly implicate the noncanonical NFκB pathway, specifically RELB (and not RELA), in directly inducing APOBEC3B transcription in response to PMA activation of PKC.

### Endogenous APOBEC3B Expression is Mediated by the PKC-NFκB Axis in Multiple Cancer Cell Lines

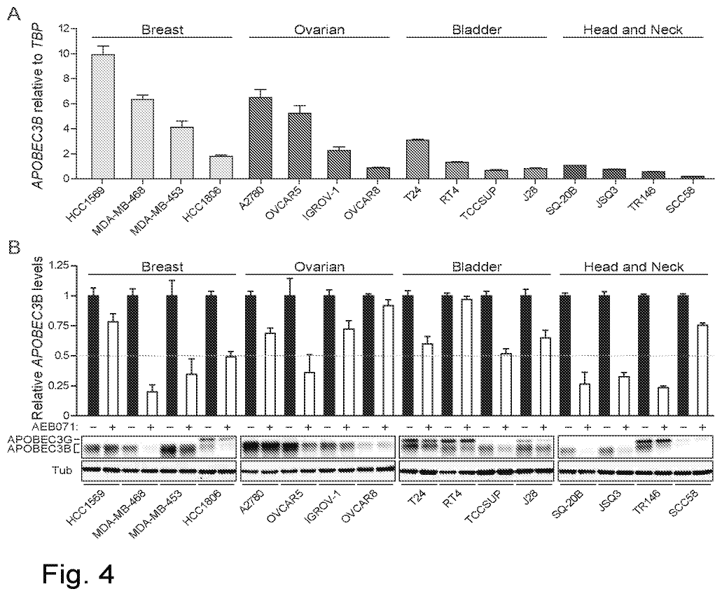

Four breast cancer cell lines, four ovarian cancer cell lines, four bladder cancer cell lines, and four head/neck cancer cell lines were analyzed to determine whether the constitutively high levels of endogenous APOBEC3B observed in many human cancer cell lines occurs through the PKC pathway. The selected cell lines expressed a 10-fold range of endogenous APOBEC3B mRNA levels (FIG. 4A).

Each line was treated for 48 hours with 10 μM AEB071, and then APOBEC3B mRNA and protein levels were quantified by RT-qPCR and immunoblotting. As above, no effects on the cell cycle or cell viability were observed (data not shown). This is important since higher concentrations of AEB071 are known to cause cell cycle perturbations and apoptosis in certain cell types. APOBEC3B mRNA levels were reduced by more than half in 7/16 cell lines, including the breast cancer cell lines MDA-MB-468, MDA-MB-453, and HCC1806, the ovarian cancer cell line OVCAR5, and the head/neck lines SQ-20B, JSQ3, and TR146 (FIG. 4B, histogram). Changes of protein levels largely mirrored the mRNA results (FIG. 4B, immunoblot). Together, these data demonstrate that the PKC axis is responsible for the constitutive upregulation of endogenous APOBEC3B in a variety of cancer cell lines representing multiple distinct cancer types.

The studies described above are the first to demonstrate that the PKC-NFκB pathway is responsible for inducing ABOBEC3B expression in breast, ovarian, bladder, and head/neck cancers. A series of experiments using the immortalized normal breast epithelial cell line MCF10A showed that the diacylglycerol analog PMA activates PKCa, which then signals through the non-canonical NFκB pathway and results in the recruitment of RELB to the APOBEC3B gene and its transcriptional activation (FIG. 6). This mechanism appears specific to APOBEC3B, as expression of the related APOBEC family members is not affected. This specificity is consistent with APOBEC3B being the only DNA deaminase family member upregulated in these and other cancer types in comparison to normal tissues. Moreover, PKC inhibitor studies with cancer cell lines indicated that the PKC-NFκB pathway may be responsible for the constitutively high levels of APOBEC3B documented previously in a large proportion of breast, ovarian, head/neck, bladder, and other cancers.

APOBEC3B overexpression and mutation signatures in cervical and head/neck cancers suggest that HPV infection might trigger an innate immune response that includes DNA deaminase upregulation. Also, infection by high-risk HPV types (not low-risk types) causes the specific upregulation of APOBEC3B, suggesting that this is not simply a gratuitous innate immune response to viral infection. Moreover, the E6 oncoprotein from high-risk types (again, not low risk) can be, all by itself, sufficient to trigger APOBEC3B upregulation. Also, the E7 oncoprotein may contribute to APOBEC3B upregulation. The mutator phenotype induced by HPV infection may fuel tumor evolution as the pattern of PI3K-activating mutations in HPV-positive tumors is biased toward cytosine mutations in APOBEC signature motifs in the helical domain of the kinase, whereas the pattern in HPV-negative tumors is split between the helical and kinase domains of the enzyme. While HPV-mediated upregulation of APOBEC3B predominantly impacts cervical and a proportion of head/neck and bladder carcinomas, other tumor types may be susceptible to similar treatment.

For example, one can locally infect a tumor with an attenuated virus or otherwise harmless/non-infectious version of a virus known to upregulate APOBEC3B. Prior to the initiation of immunotherapy, one can inject into tumors HPV fragments (e.g., full E6 protein, partial E6 protein, short E6 coding sequences, and/or other features) that tumor cells would recognize as being of HPV origin. In response to the HPV fragments, the tumor cells would upregulate APOBEC3B and the mutational load of the tumor would increase, making the tumor cells more susceptible to subsequent immunotherapy. Alternatively, one can genetically modify HPV (or other APOBEC3-upregulating virus) so that it can be used safely as a way to upregulate APOBEC3B in tumors prior to initiation of immunotherapy.

Activating the lymphotoxin-0 receptor through treatment of infected hepatocytes with bivalent or tetravalent antibodies led the nuclear translocation of both RELA and RELB and the activation of known NFκB pathway genes. These antibody treatments also led to the upregulation of APOBEC3B and to the gratuitous deamination of HBV cccDNA cytosines, viral DNA degradation, and long-term virus suppression. Thus, both the PKC and the lymphotoxin-β receptor signal transduction cascades may signal through the non-canonical RELB-dependent NFκB pathway in order to activate APOBEC3B expression. Accordingly, as described above in more detail, APOBEC3 upregulators useful for the methods described herein are not limited to PMA, but may include any compound or substance that upregulates expression of APOBEC3B, whether the APOBEC3B upregulator acts through the non-canonical PKC-NFκB pathway—e.g., PMA, an LTβR agonist, ingenol mebutate, a Smac mimetic, an IKKα activator, overexpressing P52, or cleaving NIK- or via another metabolic pathway.

Thus, this disclosure describes methods of treating a subject having a tumor. Generally, the method includes administering to a person having or at risk of having a tumor an effective amount of an APOBEC3B upregulator. As used herein, “treat” or variations thereof refer to reducing, limiting progression, ameliorating, or resolving, to any extent, the symptoms or signs related to a condition. As used herein, “symptom” refers to any subj ective evidence of disease or of a patient's condition; “sign” or “clinical sign” refers to an objective physical finding relating to a particular condition capable of being found by one other than the patient. As used herein, “ameliorate” refers to any reduction in the extent, severity, frequency, and/or likelihood of a symptom or clinical sign characteristic of a particular condition.

In some embodiments, the method can further include obtaining a sample from the tumor and determining whether tumor cells express an APOBEC3 polypeptide. In some embodiments, the APOBEC3 polypeptide can be APOBEC3B. One can determine whether cells in the sample express an APOBEC3 polypeptide by any method suitable for detection of a polypeptide by a cell. Exemplary methods include, for example, suitable forms of chromatography, electrophoresis, and/or immunoassays. In some cases, an immunoassay can employ an APOBEC3-specific antibody such as, for example, an antibody described in U.S. Provisional Patent Application No. 62/186,109, filed Jun. 29, 2015. In some embodiments, the presence of APOBEC3B in the cells of the tumor is assayed by RT-qPCR, detecting an APOBEC3B mutation through DNA sequencing, or detecting the protein itself using an APOBEC3B-specific antibody.

The APOBEC3 upregulator can be any compound or substance that upregulates expression of a member of the APOBEC3 family of enzymes. In some exemplary embodiments, the APOBEC3 upregulator is a compound or substance that upregulates APOBEC3B. As used herein, “upregulate” and variations thereof refer to the property of increasing the expression of a member of the APOBEC3 family. As used herein, “express” and variations thereof refer to the ability of a cell to transcribe a structural gene to produce an mRNA and/or then translating the mRNA to form a protein that provides a detectable biological function to the cell. Thus, “expression” of a member of the APOBEC3 family can be measured and/or described with reference to transcription of DNA to mRNA, translation of mRNA to a polypeptide, post-translational steps (e.g., modification of the primary amino acid sequence; addition of a phosphate, a carbohydrate, a lipid, a nucleotide, or other moiety to the protein; assembly of subunits; insertion of a membrane-associated protein into a biological membrane; and the like), APOBEC3 cellular activity (including mutagenesis), or any combination of the foregoing.

Exemplary APOBEC3 upregulators include, for example, PKC activators such as, for example, fumonisin B₁, phorbol esters (e.g., phorbol 12-myristate 13-acetate (PMA), 12-O-Tetradecanoylphorbol-13-acetate (TPA), phorbol-12,13-dibutyrate (PDBu)), indolactam V, a pyridazine derivative (e.g., 3-[[(2-Methylphenyl)methyl]thio]-6-(2-pyridinyl)-pyridazine (LDN/OSU-0212320)), euphohelioscopin A, prostratin, natural PKC agonists such as diacylglycerol (DAG), DAG analogs such as 1-oleoyl-2-acetyl-sn-glycerol (OAG), ingenol mebutate (ingenol 3-angelate), a bryostatin (e.g., bryostatin 1), an LTβR agonist (e.g., antibodies BS1 and CBE11), a Smac mimetic, an IKKα activator, or a viral protein such as, for example, the HPV E6 oncoprotein, the polyomavirus large T antigen, or an APOBEC3-upregulating portion or fragment thereof.

In some embodiments, PKC-NFκB activation can involve Ca²⁺, DAG, and a phospholipid such as phosphatidylserine for activation. Thus, in some embodiments in which the APOBEC3 upregulator acts through PKC-NFκB activation, the APOBEC3 upregulator may be co-administered with one or more of Ca²⁺, DAG, and/or a phospholipid as a co-upregulator.

The APOBEC3 upregulator can be administered to a subject having or at risk of having a tumor such as, for example, a tumor resulting from acute lymphoblastic leukemia (ALL), bladder cancer, breast cancer, cervical cancer, chondrosarcoma, chronic lymphocytic leukemia (CLL), esophageal cancer, head and neck cancer, kidney cancer, lung cancer, B cell lymphoma, melanoma, myeloma, osteosarcoma, ovarian cancer, pancreatic cancer, stomach cancer, thyroid cancer, uterine cancer, and uveal cancer. As used herein, the term “tumor” refers to a general state of neoplasia and does not necessarily connote a solid mass. Thus, as used herein, a tumor may be characterized as solid or as liquid. Generally, a solid tumor involves a solid mass of neoplastic cells. Generally, a liquid tumor involves neoplasias of the blood, bone marrow or the lymphatic system and do not necessarily form a solid mass.

An APOBEC3 upregulator may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with an APOBEC3 upregulator without causing any undesirable biological effects or interacting in a deleterious manner with any of the other components of the pharmaceutical composition in which it is contained.

An APOBEC3 upregulator may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), topical (e.g., intranasal, intrapulmonary, intramammary, intravaginal, intrauterine, intradermal, transcutaneous, rectally, etc.) or intratumoral. A pharmaceutical composition can be administered to a mucosal surface, such as by administration to, for example, the nasal or respiratory mucosa (e.g., by spray or aerosol).

A pharmaceutical composition may be delivered using any suitable drug delivery device or technology. In some cases, a pharmaceutical composition may be administered for systemic exposure. In other embodiments, the pharmaceutical composition may be administered for local or targeted exposure of a particular body compartment, tissue, or tumor. In some cases, a drug delivery technology can control the kinetics of release of the pharmaceutical composition to provide, for example, a pulsatile profile, a sustained or continuous profile, a delayed onset profile, or some combination of these profiles. One exemplary drug delivery approach includes liposomes loaded with a pharmaceutical composition. The liposomes can be functionalized with aptamers, peptides, and/or segments of DNA or RNA for targeting delivery to and uptake into a particular cell type or tumor. Similarly, hollow spherical nucleic acids (SNA) can carry a pharmaceutical composition into a cell through the use of ordered and functionalized oligonucleotides placed on the surface of the SNA. Degradable and non-degradable polymeric particles (e.g., microparticles and/or nanoparticles) can be loaded with the pharmaceutical composition for systemic or local drug distribution. In some embodiments, the pharmaceutical composition may be delivered using polymer micelles. Iontophoresis, ultrasound, and other forms of energy can be used to increase the permeability of a drug into a tissue or cell. In some embodiments, the pharmaceutical composition may be delivered using implantable drug delivery device. In some of these embodiments, an implantable device such as, for example, a degradable polymer depot can have a short duration of action (e.g., hours to weeks). In other embodiments, an implantable device such as, for example, a pump or a microdevice can be implanted to deliver a pharmaceutic composition over a longer (e.g., months, years, or permanently) period of time. Many longer term and permanent implants may be programmed to deliver a drug at a particular time or with a specific kinetic profile. In addition, such devices can usually be refilled with the pharmaceutical composition from time to time. One or more PKC-noncanonical NFκB axis inhibitors can be delivered by any one or any combination of drug delivery techniques.

As described above, APOBEC3B upregulation can increase the mutation rate in a cell. Having a higher mutation rate is generally undesirable. The methods described herein, however, exploit an increased mutation rate in cells in which an APOBEC3 protein has been upregulated. As a result, the methods described herein can make a cancer cell more susceptible to immunotherapy or becomes more susceptible to other cancer therapies (e.g., chemotherapy, immunotherapy, radiotherapy). One can therefore target the increase in mutational load to the appropriate cells to achieve an anti-cancer response.

One way to direct the increase in mutational load to cancer cells is to deliver the APOBEC3 upregulator locally to the tumor tissue by an intratumoral injection. Although it may not be possible to inject every tumor with the APOBEC3 upregulator, it is still possible to get a systemic anti-tumor response using this method. In cancer, a higher somatic mutational load can result in the presentation of more neoantigens by the tumor to stimulate a systemic, T-cell-driven immune response to the tumor. Once the immune system has seen the neoantigens created by the delivery of the APOBEC3 upregulator, the full magnitude of the systemic immune response can be unleashed by inhibiting an immune checkpoint with, for example, anti-CTLA-4, anti-PD-1, anti-PD-L1, or anti-CD19 monoclonal antibody therapy.

Achieving a systemic response to a tumor from the local delivery of an anti-cancer agent to a select number of tumors has been demonstrated. For example, an oncolytic virus, talimogene laherparepvec (T-VEC, Amgen, Inc., Thousand Oaks, Calif.), is a modified herpes virus that can be injected locally into tumors in melanoma patients. Evidence of a systemic anti-tumor response was seen in a Phase III clinical trial where 11% of patients experienced complete disappearance of melanoma throughout the body, including tumors that had not been directly injected with the drug. Other exemplary viral vectors for delivery may be based upon a retrovirus, a lentivirus, an adenovirus, an adeno-associated virus, a pox virus, an alphavirus, a herpes virus, a measles virus, an influenza virus, etc.

Another way to deliver the APOBEC3 upregulator is to enclose it in liposomes that are functionalized with aptamers, peptides, and/or segments of DNA or RNA for targeting delivery to, and uptake into, a particular cell type or tumor. These functionalized liposomes can be delivered systemically because the liposomes will only be taken up by the tumor or other cells having the targeted receptor on the surface.

Finally, because APOBEC3B is preferentially overexpressed in tumor cells, it may be possible to deliver an APOBEC3B upregulator systemically and observe the APOBEC3B upregulation and increased mutation only in the diseased target cells. The healthy cells where APOBEC3B is not overexpressed will not be affected by the APOBEC3B upregulator.

Thus, an APOBEC3 upregulator may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the APOBEC3 upregulator into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of APOBEC3 upregulator administered can vary depending on various factors including, but not limited to, the specific APOBEC3 upregulator, the weight, physical condition, and/or age of the subject, and/or the route of administration. Thus, the absolute weight of APOBEC3 upregulator included in a given unit dosage form can vary widely, and depends upon factors such as the species, age, weight and physical condition of the subject, and/or the method of administration. Accordingly, it is not practical to set forth generally the amount that constitutes an amount of APOBEC3 upregulator effective for all possible applications. Those of ordinary skill in the art, however, can readily determine the appropriate amount with due consideration of such factors.

For example, certain APOBEC3 upregulators may be administered at the same dose and frequency for which the drug has received regulatory approval. In other cases, certain APOBEC3 upregulator may be administered at the same dose and frequency at which the drug is being evaluated in clinical or preclinical studies. One can alter the dosages and/or frequency as needed to achieve a desired level of APOBEC3 upregulation. Thus, one can use standard/known dosing regimens and/or customize dosing as needed.

In some embodiments, the method can include administering sufficient APOBEC3 upregulator to provide a dose of, for example, from about 100 ng/kg to about 50 mg/kg to the subject, although in some embodiments the methods may be performed by administering APOBEC3 upregulator in a dose outside this range. In some of these embodiments, the method includes administering sufficient APOBEC3 upregulator to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184. In some embodiments, the method can include administering sufficient APOBEC3 upregulator to provide a dose of, for example, from about 0.01 mg/m² to about 10 mg/m².

In some embodiments, the APOBEC3 upregulator may be administered, for example, from a single dose to multiple doses per week, although in some embodiments the method can be performed by administering the APOBEC3 upregulator at a frequency outside this range. In certain embodiments, the APOBEC3 upregulator may be administered from about once per month to about five times per week.

In some embodiments, the APOBEC3 upregulator can be co-administered with a second therapy. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, resection of at least a portion of a solid tumor, etc.) persists at the treatment site until one or more additional components are administered to the treatment site. Thus, a co-administered combination can, in certain circumstances, include components that never exist in a chemical mixture with one another.

One example of sequential co-administration is an exemplary treatment regimen in which an APOBEC3 upregulator is administered to a subject, the APOBEC3 upregulator is given sufficient time to increase mutation in tumor cells, and then providing immunotherapy to the subject. In some embodiments, the immunotherapy can include a treatment that blocks an immune checkpoint inhibitor—i.e., the treatment stimulates the immune system by inhibiting an inhibitor of the immune system. Exemplary immune checkpoint inhibitors that can be the target of immunotherapy include CTLA-4, PD-1, and/or PD-L1. Exemplary drugs that target immune system inhibitors include, for example, ipilumimab (e.g., YERVOY, Bristol-Myers Squibb Co., New York, N.Y.), nivolumab (e.g., OPDIVO, Bristol-Myers Squibb Co., New York, N.Y.), or pembrolizumab (KEYTRUDA, Merck & Co., Inc., Kenilworth, N.J.), atezolizumab (TECENTRIQ, Genentech Inc., South San Francisco, Calif.), durvalumab (AstraZeneca plc, London, United Kingdom), tremelimumab (AstraZeneca plc, London, United Kingdom), or inebilizumab (AstraZeneca plc, London, United Kingdom).

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Cell Lines

MCF10A (ATCC CRL-10317), HCC1569 (ATCC CRL-2330, and MDA-MB-468 (ATCC HTB-132) were purchased from the American Type Culture Collection (ATCC) and cultured as recommended. A2780 and OVCAR5 were obtained from Dr. Scott Kaufmann (Mayo Clinic, Rochester, Minn.) and cultured as reported (Leonard et al., 2013, Cancer Res 73(24):7222-7231). SQ20B and JSQ3 were obtained from Dr. Mark Herzberg (University of Minnesota-Twin Cities, Minneapolis, Minn.) and cultured at 37° C. with 5% CO₂ in DMEM/F12 with 10% fetal bovine serum, penicillin, streptomycin, and 400 ng/mL hydrocortisone. These and other cell lines are listed in Table 1.

TABLE 1

| Cell Line | Type | Growth Conditions^{a} | Source |
|---|---|---|---|
| 293T | Fibroblast | DMEM, 10% FBS, Pen-Strep | Dr. Michael Malim, |
| | | | Kings College |
| | | | London |
| HeLa | Cervical Cancer | RPMI, 10% FBS, Pen-Strep | |
| MCF7L | Breast Cancer | IMEM, 5% FBS, Pen-Strep, 11.2 5 nM | Dr. Doug Yee, |
| | | human insulin | University of |
| | | | Minnesota |
| N/TERT | Keratinocyte | K-SFM, Pen-Strep, 0.3 mM CaCl₂, 0.2 ng/ml | Dr. Peter Howley, |
| | | EGF, 25 μg/ml BPE | Harvard University |
| NIKS | Keratinocyte | E media, 5% FBS, Pen-Strep, 24 μg/ml | Dr. Paul Lambert, |
| | | adenine, 8.4 μg/ml cholera toxin, 10 ng/ml | University of |
| | | EGF, 400 ng/ml hydrocortisone, 5 μg/ml | Wisconsin, Madison |
| | | insulin | |
| MCF10A | Epithelial | MEGM without gentimicin, Pen-Strep, | ATCC CRL-10317 |
| | | 10 μg/ml cholera toxin | |
| HCC1569 | Breast Cancer | RPMI, 10% FBS, Pen-Strep | ATCC CRL-2330 |
| MDA-MB- | Breast Cancer | Liebovitz's L-15, 10% FBS, Pen-Strep | ATCC HTB-132 |
| 468 | | | |
| MDA-MB- | Breast Cancer | Liebovitz's L-15, 10% FBS, Pen-Strep | ATCC HTB-131D |
| 453 | | | |
| HCC1806 | Breast Cancer | RPMI, 10% FBS, Pen-Strep | ATCC CRL-2335 |
| A2780 | Ovarian Cancer | RPMI, 10% FBS, Pen-Strep | Dr. Scott Kaufmann, |
| | | | Mayo Clinic |
| OVCAR5 | Ovarian Cancer | RPMI, 10% FBS, Pen-Strep | Dr. Scott Kaufmann, |
| | | | Mayo Clinic^{c} |
| IGROV-1 | Ovarian Cancer | McCoy's 5A, 10% FBS, Pen-Strep | Dr. Scott Kaufmann, |
| | | | Mayo Clinic |
| OVCAR8 | Ovarian Cancer | RPMI, 10% FBS, Pen-Strep | Dr. Scott Kaufmann, |
| | | | Mayo Clinic |
| T24 | Bladder Cancer | McCoy's 5A, 10% FBS, Pen-Strep | ATCC HTB-4 |
| RT4 | Bladder Cancer | McCoy's 5A, 10% FBS, Pen-Strep | ATCC HTB-2 |
| TCCSUP | Bladder Cancer | MEM in EBSS, 10% FBS, Pen-Strep, NEAA, | ATCC HTB-5 |
| | | 1 mM sodium pyruvate | |
| J28 | Bladder Cancer | MEM in EBSS, 10% FBS, Pen-Strep, NEAA, | ATCC |
| | | 1 mM sodium pyruvate | |
| SQ-20B | Head/neck | DMEM/F12, 10% FBS, Pen-Strep, 400 ng/mL | Dr. Mark Herzberg, |
| | cancer | hydrocortisone | University of |
| | | | Minnesota |
| JSQ3 | Head/neck | DMEM/F12, 10% FBS, Pen-Strep | Dr. Mark Herzberg, |
| | cancer | | University of |
| | | | Minnesota^{b} |
| TR146 | Head/neck | DMEM/F12, 10% FBS, Pen-Strep, 400 ng/mL | Dr. Mark Herzberg, |
| | cancer | hydrocortisone | University of |
| | | | Minnesota |
| SCC58 | Head/neck | DMEM/F12, 10% FBS, Pen-Strep, 400 ng/mL | Dr. Mark Herzberg, |
| | cancer | hydrocortisone | University of |
| | | | Minnesota |

^{a}Abbreviations: FBS: fetal bovine serum; Pen-Strep: 100 U/mL penicillin and streptomycin; EGF: epidermal growth factor; BPE: bovine pituitary extract; EBSS: Earl's balanced salt solution; NEAA: 1x non-essential amino acids.
^{b}Weichselbaum et al., 1988, Int. J. Radiat Oncol. Biol. Phys. 15: 575-579.
^{c}Monks et al., 1991, J. Natl. Cancer Inst. 83: 757-766.

### PMA Induction and PKC-NFκB Inhibitors

For induction experiments, 2.5×10⁵ cells were plated in a 6-well plate 1 day prior to drug treatment. PMA was then added to the media and incubated at 37° C. with 5% CO₂ for six hours unless otherwise indicated. Cells were harvested, RNA was extracted, and RT-qPCR was performed as previously reported (Refsland et al. 2010, Nucleic Acids Res 38:4274-4284). For experiments utilizing inhibitors, cells were pretreated with inhibitors 30 minutes prior to PMA induction (25 ng/mL). PMA (Thermo Fisher Scientific, Inc., Waltham, Mass.), cyclohexamide (Acros Organics, Thermo Fisher Scientific, Inc., Waltham, Mass.), G66983 (Cayman Chemical co., Ann Arbor, Mich.), LY294002 (EMD Chemicals, Merck KGaA, Darmstadt, Germany), U0126 (EMD Chemicals, Merck KGaA, Darmstadt, Germany), AEB071 (Medchemexpress, Monmouth Junction, N.J.), Go6976 (Enzo Life Sciences, Inc., Farmingdale, N.Y.), BAY 11-7082 (R&D Systems, INc., Minneapolis, Minn.), and MG132 (Thermo Fisher Scientific, Inc., Waltham, Mass.) were stored as recommended. NIKi/Compound 31 was synthesized as described (Li et al., 2013, Bioorganic & Medicinal Chemistry Letters 23:1238-1244).

### Immunoblotting

The development and validation of the rabbit monoclonal antibody (mAb) against APOBEC3B is as described elsewhere (U.S. Provisional Patent Application No. 62/186,109, filed Jun. 29, 2015). The mAb used (referred to as 5210-87-13) effectively binds endogenous APOBEC3B in a variety of assays. The anti-tubulin antibody was obtained from Covance Inc., Princeton, N.J.

### Deaminase Activity Assays

Deaminase activity assays were performed as previously reported (Vieira et al., 2014, mBio 5(6):e02234-14). In short, 4 pmol of a fluorescently labeled oligo with a single target cytosine (5′-ATTATTATTATTCAAATGGATTTATTTATTTATTTATTTATTT-fluorescein, SEQ ID NO:2) was treated with cell extract containing 0.025 U/rxn UDG (New England BioLabs, Inc., Ipswich, Mass.), UDG buffer, and 1.75 U/rxn RNase A (Qiagen, Hilden, Germany) for two hours. Abasic sites were cleaved by treatment with 100 mM NaOH at 95° C. for 10 minutes. Substrate was separated from product using 15% TBE-urea gel electrophoresis. Gels were scanned using a FujiFilm Image Reader FLA-7000.

### RNA Sequencing Experiments

Two sets of MCF10A cells in duplicate were treated every eight hours with media supplemented with PMA or DMSO for 48 hours. At 48 hours, RNA was extracted using an RNeasy Mini Kit (Qiagen, Hilden, Germany). Total RNA was submitted to the University of Minnesota Genomics Center for sequencing on the Illumina HiSeq 2000 platform. Raw reads were analyzed using both DESeq2 (Love et al., 2014, Genome Biol. 15:550) and the Tuxedo suite (Trapnell et al., 2012, Nat Protoc. 7:562-578) to identify changes in mRNA expression in PMA treated versus untreated cells.

### Chromatin Immunoprecipitation Experiments

MCF10A cells were treated with either DMSO or 25 ng/mL PMA for two hours. Cross-linking was performed with 1% formaldehyde for 10 minutes at room temperature and quenched with 150 mM glycine. Cells were then lysed in Farnham Lysis Buffer at 4° C. for 30 minutes. Nuclei were pelleted, resuspended in RIPA Buffer, and sonicated (BIORUPTOR Pico, Diagenode S.A., Liege, Belgium) to generate approximately 600 bp DNA fragments. Immunoprecipitations were done using Protein G Dynabeads (Invitrogen, Thermo Fisher Scientific, Inc., Waltham, Mass.) and 2 Lg antibody per sample. Samples were washed in 1 mL low salt wash buffer, 1 mL high salt wash buffer, 1 mL LiCl wash buffer, and eluted at 65° C. for 30 minutes. Samples were reverse cross-linked using 200 mM NaCl and treated with Proteinase K for 12 hours at 65° C. DNA was purified using a ChIP DNA Clean and Concentrator Kit (Zymo Research Corp., Irvine, Calif.) and qPCR was performed with SYBR Green master mix (Roche Diagnostics USA, Indianapolis, Ind.) on a Roche LightCycler 480. Values represent the percentage of input DNA immunoprecipitated (IP DNA) and are the average of three independent qPCR reactions.

ChIP reagents are listed in Table 2.

TABLE 2
ChIP reagents

| Category | Reagent | Description |
|---|---|---|
| Antibody | Rabbit IgG | sc-2027, Santa Cruz Biotechnology, |
| | | Inc., Dallas, TX |
| Antibody | RNA Pol II (Ser 5) | ab5131, Abcam plc, Cambridge, |
| | | United Kingdom |
| Antibody | Rel A (p65) | sc-372, Santa Cruz Biotechnology, |
| | | Inc., Dallas, TX |
| Antibody | Rel B | sc-226, Santa Cruz Biotechnology, |
| | | Inc., Dallas, TX |
| Buffer | Farnham Lysis buffer | 5 mM PIPES pH 8 |
| | | 85 mM KCl |
| | | 0.5% Nonidet P-40 |
| | | 1x EDTA-free Protease Inhibitor |
| | | Cocktail (Roche) |
| Buffer | RIPA buffer | 50 mM Tris-HCl pH 8 |
| | | 150 mM NaCl |
| | | 5 mM EDTA |
| | | 1% Nonidet P-40 |
| | | 0.5% deoxycholate |
| | | 0.1% SDS |
| | | 1x EDTA-free Protease Inhibitor |
| | | Cocktail (Roche) |
| Buffer | Low salt wash buffer | 20 mM Tris-HCl pH 8 |
| | | 150 mM NaCl |
| | | 2 mM EDTA |
| | | 0.1% SDS |
| | | 1% Triton X-100 |
| Buffer | High salt wash buffer | 20 mM Tris-HYCl pH 8 |
| | | 500 mM NaCl |
| | | 2 mM EDTA |
| | | 0.1% SDS |
| | | 1% Triton X-100 |
| Buffer | LiCl wash buffer | 20 mM Tris-HCl pH 8 |
| | | 0.5M LiCl |
| | | 1% Nonidet P-40 |
| | | 1% deoxycholate |
| | | 1 mM EDTA |
| Buffer | Elution buffer | 100 mM NaHCO₃ |
| | | 1% SDS |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of treating a subject having a tumor, the method comprising:
administering to the subject an amount of an APOBEC3 upregulator effective to increase mutagenesis in cells of the tumor.

2. The method of claim 1 wherein the APOBEC3 upregulator is co-administered with an immunotherapy.

3. The method of claim 2 wherein the APOBEC3 upregulator is administered prior to the immunotherapy.

4. The method of claim 2 wherein the immunotherapy comprises administering to the subject a therapeutic molecule that blocks an immune checkpoint.

5. The method of claim 4 wherein the immune checkpoint inhibitor comprises an anti-CTLA-4 antibody, an anti-PD-1 antibody, or an anti-PD-L1 antibody.

6. The method of claim 4 wherein the immunotherapy comprises administering to the subject an effective amount of ipilumimab, nivolumab, pembrolizumab, atezolizumab, durvalumab, tremelimumab, or inebilizumab.

7. The method of claim 1, further comprising:
obtaining a sample from the tumor; and
determining whether tumor cells express an APOBEC3 mRNA, an APOBEC3 polypeptide, or an APOBEC3 mutation signature.

8. The method of claim 7 wherein the APOBEC3 mRNA comprises an APOBEC3B mRNA, the APOBEC3 polypeptide comprises an APOBECB polypeptide, or the APOBEC3 mutation signature comprises an APOBEC3B mutation signature.

9. The method of claim 1, wherein the APOBEC3 upregulator is administered intratumorally.

10. The method of claim 1, wherein the APOBEC3 upregulator is administered systemically.

11. The method of claim 1, wherein the APOBEC3 upregulator is administered via functionalized liposomes.

12. The method of claim 1, wherein the APOBEC3 upregulator is administered via a virus-based vector.
